(19) United States
(12) Patent Application Publication
(10) Pub. No.: US 2020/0360242 A1
(43) Pub. Date: Nov. 19, 2020

(54) Oral Care Gel

(71) Applicant: Colgate-Palmolive Company, New York, NY (US)

(72) Inventors: Mahmoud HASSAN, Somerset, NJ (US); Lauren EVANS, Piscataway, NJ (US)

(73) Assignee: Colgate-Palmolive Company, New York, NY (US)

(21) Appl. No.: 15/779,755

(22) PCT Filed: Dec. 2, 2015
(86) PCT No.: PCT/US2015/063443

§ 371 Date: May 29, 2018

(51) Int. Cl. A61K 8/04; A61K 8/22; A61K 8/89; A61K 8/81; A61K 8/60; A61Q 11/00

(52) U.S. Cl. CPC A61K 8/042; A61K 8/22; A61K 8/89; A61K 8/8111; A61K 2800/594; A61K 8/60; A61Q 11/00; A61K 2800/5422; A61K 8/8182

(57) ABSTRACT

The invention provides an orally acceptable tooth whitening gel wherein the gel comprises a hydrogen peroxide source, a silicone compound, a porous cross-linked polymer, and a nonionic surfactant, and exhibits a Herschel-Bulkley yield stress of 10 to 230 dynes/cm², a Herschel-Bulkley viscosity of 3 to 500 poise, and a Herschel-Bulkley rate index of 0.4 to 0.6, the specific viscosity permitting efficient application of the gel, as well as methods of using the same.

### FIELD OF THE INVENTION

The present invention relates to improved tooth whitening gels.

### BACKGROUND OF THE INVENTION

Oral care compositions comprising tooth whitening agents have been known for many years and include liquids, gels, tablets, rinses, strips, trays, pens and other applicator devices. However, applying oral care products to teeth can present difficulties. For example, application of these compositions can be messy, as the whitening agent is capable of dripping into sensitive mucosal surfaces such as the lips, gums, and tongue, where it may cause considerable irritation. In addition, it may be difficult to apply the whitening agent in a precise way or to apply it to difficult to reach parts of the teeth.

Hydrogen peroxide is a common whitening agent used in many current tooth whitening devices, such as strips, trays, gels and pens. It is an unstable compound and in the presence of water it will slowly undergo decomposition, losing its whitening capacity. While tooth whitening pens are known in the art, they face two main difficulties. First, it is difficult to formulate a whitening composition with the necessary viscoelastic properties to be used with a pen applicator. For instance, the material extruded from the pen, typically a gel, must strike a fine balance of viscoelastic properties so that it is easy to dispense and to apply to the teeth, and so that it sticks to the teeth sufficiently to have a useful whitening effect, but that it is spreads easily over the surface of the teeth to ensure a uniform and complete whitening effect. The second difficulty is in developing a formulation that has the desired viscoelastic properties yet also retains sufficiently long lasting activity for the hydrogen peroxide active ingredient. One solution to ensure the stability of hydrogen peroxide in products of this type has often been to use hydrogen-peroxide polymer complexes (for example, polyvinylpyrrolidone (PVP)-hydrogen peroxide complex) as the hydrogen peroxide source. This polymeric hydrogen peroxide compound is stable in non-aqueous environments, but upon exposure to water, the complex begins to break down releasing active hydrogen peroxide. Thus, products using PVP-H₂O₂ and similar actives are usually formulated to be substantially free of water, for example, by using a large proportion of hydrophobic ingredients. Typically, such formulations may consist of up to 70% by weight or more of hydrophobic components. However, the drawback to this approach is that in order for the hydrogen peroxide to be released to exert its bleaching effect on the teeth, water must be admitted into the formulation that is spread over the teeth. With a highly hydrophobic formulation, water does not readily penetrate the gel, and this results in poor bleaching efficiency due to insufficient breakdown of the PVP-H₂O₂ complex.

There is thus an unmet market need for a product that can apply oral hydrogen peroxide based whitening agent to the teeth, without messiness or difficulty of use and application, and employing an efficient and stable formulation of active agent.

### BRIEF SUMMARY OF THE INVENTION

The present disclosure provides a hydrogen peroxide-based whitening gel with improved rheological properties and improved stability and efficacy. The inventors have unexpectedly found that the inclusion of a non-ionic surfactant in the whitening gel formulation results in greatly improved release of hydrogen peroxide and more efficient whitening.

The gels of the present disclosure are suitable for application to the teeth with a pen-type device, having a viscosity which permits controlled application and retention on the teeth for a sufficient period to allow adequate whitening, and further permits the gel to be dispensed easily and spread evenly on the teeth.

Characterization and optimization of the viscosity of such non-Newtonian fluids is complex. The Herschel-Bulkley (HB) model is a generalized model of a non-Newtonian fluid, in which the strain experienced by the fluid is related to the stress in a non-linear way. Three parameters characterize this relationship: the consistency k, the flow index n, and the yield shear stress ro. The consistency is a simple constant of proportionality. The flow index measures the degree to which the fluid is shear-thinning or shear-thickening. Finally, the yield stress quantifies the amount of stress that the fluid may experience before it yields and begins to flow.

The gels described herein are shear-thinning, meaning that the viscosity of the gel decreases in accordance with the Herschel-Bulkley Model as more force is applied (shear stress). The Herschel-Bulkley Model provides a profile of the rheology of the gels at different shear stress. Through empirical evaluation of a number of gels, it is determined that the gels in accordance with the present disclosure should preferably exhibit a Herschel-Bulkley yield stress of 10 to 230 dynes/cm², e.g., 30 to 45 dynes/cm², a Herschel-Bulkley viscosity of 3 to 500 poise, e.g., 30 to 45 poise, and a Herschel-Bulkley rate index of 0.4 to 0.6, e.g. 0.5 to 0.6.

The oral care systems of the invention thus comprise a gel in a pen dispenser, the dispenser comprising a chamber which permits dispensing of a measured amount of the gel to an applicator head, e.g. a doe foot or brush applicator head, wherein the gel exhibits a Herschel-Bulkley yield stress of 10 to 230 dynes/cm², e.g., 30 to 45 dynes/cm², a Herschel-Bulkley viscosity of 3 to 500 poise, e.g., 30 to 45 poise, and a Herschel-Bulkley rate index of 0.4 to 0.6, e.g. 0.5 to 0.6.

Using a gel having an optimized viscosity in a pen applicator device allows for more controlled application and reduces the level of active agent required in the formulation, thereby making the application more efficient, more effective, and less messy than prior art approaches.

Further areas of applicability of the present invention, including methods of making and using the gels for use in the invention, will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE INVENTION

The following description of the preferred embodiment(s) is merely exemplary in nature and is in no way intended to limit the invention, its application, or uses.

In one embodiment, the gel in the dispenser is an orally acceptable tooth whitening gel (Gel 1) comprising:
(i) a hydrogen peroxide source,
(ii) a silicone compound,
(iii) a porous cross-linked polymer, and
(iv) a nonionic surfactant,
   the gel having a Herschel-Bulkley yield stress of 10 to 230 dynes/cm², e.g., 30 to 45 dynes/cm², a Herschel-Bulkley viscosity of 3 to 500 poise, e.g., 30 to 45 poise, and a Herschel-Bulkley rate index of 0.4 to 0.6, e.g. 0.5 to 0.6.

For example, the invention provides in various embodiments
1.1. Gel 1, wherein the hydrogen peroxide source is a hydrogen peroxide-polymer complex.
1.2. Gel 1.1 or 1.2, wherein the hydrogen peroxide source is a polyvinylpyrrolidone-hydrogen peroxide complex
1.3. Gel 1.2, wherein the polyvinylpyrrolidone-hydrogen complex is a cross-linked polyvinylpyrrolidone-hydrogen peroxide complex.
1.4. Any of Gels 1 or 1.1-1.2, wherein the gel contains 0.1-10% by weight of hydrogen peroxide, e.g., 0.5-10% by weight, or 1-5% by weight, or 2-5% by weight, or 3-5% by weight, or 4-5% by weight, or about 4.5% by weight of hydrogen peroxide.
1.5. Any of the foregoing gels, wherein the silicone compound comprises a silicone polymer, silicone adhesive, silicone gum, silicone wax, silicone elastomer, silicone fluid, silicone resin, silicone powder, or mixture thereof, as these terms are defined in U.S. Pat. No. 8,568,695.
1.6. Any of the foregoing gels, wherein the silicone compound comprises a polydiorganosiloxane.
1.7. Any of the foregoing gels, wherein the silicone compound has a viscosity at 25° C. of 500,000 cSt (centiStokes) to 50,000,000 cSt.
1.8. Any of the foregoing gels, wherein the silicone compound is a polydimethylsiloxane.
1.9. Any of the foregoing gels, wherein the silicone compound is present in the composition in an amount of 10 to 50% by weight, e.g., 20-40%, or about 30%.
1.10. Any of the foregoing gels, further comprising a hydrophilic organic polymer.
1.11. Gel 1.10, wherein the hydrophilic organic polymer is selected from a polyethylene glycol, a nonionic polymer of ethylene oxide, a block copolymer of ethylene oxide and propylene oxide, a carboxymethylene polymer, polyvinylpyrrolidone, and mixtures thereof.
1.12. Gel 1.11, wherein the hydrophilic organic polymer is polyvinylpyrrolidone (e.g., cross-linked polyvinylpyrrolidone).
1.13. Any of the foregoing gels, wherein the nonionic surfactant is selected from a polyoxyethylene sorbitan monolaurate (polysorbate) or a poly(oxyethylene)-poly(oxypropylene) block copolymers (poloxamers).
1.14. Gel 1.13, wherein the nonionic surfactant is a Polysorbate 20 or Polysorbate 80.
1.15. Any of the foregoing gels, wherein the nonionic surfactant is present at 0.01 to 10% by weight of the composition, e.g., 0.05 to 5%, or 0.15 to 1%, or 0.5 to 1%, or 0.5 to 4%, or 0.5 to 3%, or 0.5 to 2.5%, or 1 to 2.5%, or 1 to 2%, or about 1%, by weight of the composition.
1.16. Any of the foregoing gels, further comprising an alkali metal pyrophosphate (e.g. tetrasodium or tetrapotassium pyrophosphate) or an alkali metal tripolyphosphate (e.g. potassium or sodium tripolyphosphate).
1.17. Any of the foregoing gels, further comprising sodium tripolyphosphate.
1.18. Gel 1.16 or 1.17, wherein the alkali metal pyrophosphate or alkali metal tripolyphosphate (e.g. sodium tripolyphosphate or tetrasodium pyrophosphate) is present at 0.1 to 5% by weight of the composition, e.g., from 0.5 to 4%, or from 1 to 3%, or about 2% by weight of the composition.
1.19. Any of the foregoing gels, wherein the porous cross-linked polymer comprises at least one polymerized polyunsaturated monomer chosen from acrylate and methacrylate or where the porous cross-linked polymer comprises a polyitaconate, or where the porous cross-linked polymer comprises a dimethiconol cross polymer.
1.20. Any of the foregoing gels, wherein the porous cross-linked polymer comprises a dimethiconol cross-polymer, for example, dimethiconol/silsesquioxane copolymer, trimethylsiloxysilicate/dimethiconol cross-polymer, dimethiconol/acrylate copolymer.
1.21. Any of the foregoing gels, wherein the porous cross-linked polymer has a BET pore volume of 0.1 to 0.3 cc/g.
1.22. Any of the foregoing gels, wherein the silicone compound is sorbed onto the porous cross-linked polymer.
1.23. Gel 1.22, wherein the silicone compound is sorbed at an amount of 50-95% onto the porous cross-linked polymer, by weight of the combination of silicone compound and polymer, e.g., 70% to 90%.
1.24. Any of the foregoing gels, further comprising a hydrophobic adhesion agent (e.g., mineral oil, petrolatum, liquid paraffin, polyethylene waxes, silicone polymers, and PVP/vinyl acetate copolymers).
1.25. Gel 1.24, wherein the hydrophobic adhesion agent comprises from 1% to 50% by weight of the composition, e.g., from 10-40% by weight, or from 20%-30% by weight, or about 30% by weight.
1.26. Any of the foregoing gels, wherein the elastic modulus (G′) is 25 to 1750 dyne/cm², e.g., 250 to 400 dyne/cm².
1.27. Any of the foregoing gels, wherein the viscous modulus (G″) is 20 to 750 dyne/cm², e.g., 120 to 180 dyne/cm².
1.28. Any of the foregoing gels wherein the critical stress is 2.5 to 15 dyne/cm², e.g, 4 to 6 dyne/cm².
1.29. Any of the foregoing gels wherein the ratio of the elastic modulus to the viscous modulus (G′/G″) is 1-3, e.g., 1.5 to 2.3, e.g., about 2.
1.30. Any of the foregoing gels further comprising a thickening agent selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth and combinations thereof.
1.31. Any of the foregoing gels further comprising a thickening agent selected from homopolymers of acrylic acid cross-linked with an alkyl ether of pentaerythritol or an alkyl ether of sucrose, and carbomers.
1.32. Any of the foregoing gels further comprising a thickening agent selected from copolymers of lactide and glycolide monomers, the copolymer having the molecular weight in the range of from about 1,000 to about 120,000 (number average).
1.33. Any of the foregoing gels further comprising a thickener selected from cellulose derivatives (for example carboxymethyl cellulose), polysaccharide gums (for example xanthan gum or carrageenan gum), and combinations thereof.
1.34. Any of the foregoing gels further comprising 0.2-1.5% xanthan gum and 0.2-3% carboxymethyl cellulose, by weight of the composition.
1.35. Any of the foregoing gels further comprising one or more humectants present in a total amount of 1% to 50%, e.g., 2% to 25%, or 5% to 15% by weight of the composition.
1.36. Gel 1.32, wherein the humectants are selected from glycerin, sorbitol, xylitol, and combinations thereof.
1.37. Any of the foregoing gels further comprising flavorings, e.g. saccharin, mint flavor, and combinations thereof.
1.38. Any of the foregoing gels further comprising a fluoride ion source, e.g. sodium fluoride, e.g., 0.075-0.15%, e.g., 0.11%, by weight of the composition.

Gels comprising a silicone compound, a porous cross-linked polymer, and optionally a hydrophilic organic polymer are disclosed in U.S. Pat. No. 8,568,695, the contents of which are hereby incorporated herein by reference in its entirety.

Silicone compounds useful for the present disclosure include, but are not limited to, silicone polymers, silicone adhesives, silicone gums, silicone waxes, silicone elastomers, silicone fluids, silicone resins, silicone powders, and mixtures thereof.

Silicone gums useful herein include high molecular weight polydiorganosiloxanes having a viscosity, at 25° C., of 500,000 cSt up to 50,000,000 cSt (centiStokes). Such silicone gums include those polydiorganosiloxanes with a weight average molecular weight of greater than 500,000. The polysiloxane gums for use herein can be linear or cyclic, and branched or unbranched. Substituents may have any structure as long as the resulting polysiloxanes are hydrophobic, are neither irritating, toxic nor otherwise harmful when applied to the oral cavity, and are compatible with the other components of the composition. Specific examples of suitable siloxane gums include polydimethylsiloxane, methylvinylsiloxane, polydimethylsiloxane/methylvinylsiloxane copolymer, poly(dimethylsiloxane, diphenyl, methyvinylsiloxane) copolymer and mixtures thereof. Silicone gums include those commercially available and marketed by General Electric. Silicone waxes include cosmetic waxes and silky waxes.

Polysiloxane fluids useful herein include those with a viscosity, at 25° C., of 1 cSt to 1000 cSt, or 2 cSt to 500 cSt, or 20 cSt to 400 cSt. Polysiloxane fluids for use herein can be linear or cyclic, and can be substituted with a wide variety of substituents (including as described above). In certain embodiments, substituents include methyl, ethyl and phenyl substituents. Suitable polysiloxane fluids include linear polysiloxane polymers such as dimethicone and other low viscosity analogues of the polysiloxane materials, in certain embodiments having a viscosity, at 25° C., of 200 cSt or less and cyclomethicone, and other cyclic siloxanes having for example a viscosity, at 25° C., of 200 cSt or less. Other fluids include polysiloxane polyether copolymers and hydroxy terminated polydimethyl-siloxane fluid (e.g., Dow Corning ST-DIMETHICONOL™ 40, Dow Corning SGM 36, SGM3). Commercial examples of materials that are suitable for use herein include DC200 series fluids marketed by Dow-Corning Corporation and the AK Fluid series marketed by Wacker-Chemie GmbH, Munchen, Germany. High molecular silicone resins with a polysiloxane blend may also be used including powdered trimethylsiloxysilicate, for example, Dow Corning 593 fluid, Wacker Belsil TMS 803.

Suitable clastomeric silicone powders can be used having a particle size of 1 to 15 μm, for example dimethicone/vinyl dimethicone cross polymers. Additionally, in certain embodiments, non-ionic emulsions containing 30% dimethicone can be used.

As referred to herein, a “porous cross-linked polymer” is a particulate polymer material which is operable to sorb a silicone compound. The term “sorb” refers to the “sorptive” (or “sorption”) capability of the polymer particles to adsorb, absorb, complex, or otherwise retain a silicone compound. As used herein, “porous” refers to the presence of voids or interstices between cross-linked polymers that increases the overall surface area of the polymer beyond a solely perimeter measurement. Without limiting the mechanism, function or utility of the present disclosure, it is to be understood that in some embodiments the composite comprises polymeric particulates having a non-smooth surface and an irregular polymeric matrix in which the silicone compound is retained. The chemical and physical characteristics of the particulate hinder the release of the silicone compound from the polymer particulates, and in some embodiments provide sustained release of the silicone compound. In various embodiments, the polymer comprises porous particulates having a BET pore volume (Brunauer, Emmett and Teller method) of 0.05 to 0.3 cc/g, optionally 0.1 to 0.2 cc/g, optionally 0.14 to 0.16 cc/g.

In one embodiment, the cross-linked polymer is the polymerization product of at least one, and in other embodiments at least two, monomers having at least two unsaturated bonds (hereinafter referred to as “polyunsaturated” monomers), the monomers being polymerized including no more than 40% by weight, and in other embodiments less than 9% by weight, total monomer weight of monounsaturated co-monomers. The polyunsaturated monomers are selected from polyacrylates, polymethacrylates, polyitaconates and mixtures thereof. Included are polyacrylates, -methacrylates, or -itaconates of: ethylene glycol, propylene glycol; di-, tri-, tetra-, or poly-ethylene glycol and propylene glycol; trimethylol propane, glycerin, erythritol, xylitol, pentaerythritol, dipentacrythritol, sorbitol, mannitol, glucose, sucrose, cellulose, hydroxyl cellulose, methyl cellulose, 1,2 or 1,3 propanediol, 1,3 or 1,4 butanediol, 1,6 hexanediol, 1,8 octanediol, cycloliexanediol, cyclohexanetriol, and mixtures thereof. Similarly, bis(acrylamido or methacrylamido) compounds can be used. These compounds are, for example, methylene bis(acryl or methacryl)amide, 1,2 dihydroxy ethylene bis(acryl or methacryl)amide, hexamethylene bis(acryl or methacryl)amide. In one embodiment, the polyunsaturated monomer is polymethacrylate.

Another group of useful monomers include di or poly vinyl esters, such as divinyl propylene urea, divinyl-oxalate, -malonate, -succinate, -glutamate. -adipate, -sebacate, -maleate, -fumerate, -citraconate, and -mesaconate. Other suitable polyunsaturated monomers include divinyl benzene, divinyl toluene, diallyl tartrate, allyl pyruvate, allyl maleate, divinyl tartrate, triallyl melamine, N,N′-methylene bis acrylamide, glycerine dimethacryl ate, glycerine trimethacrylate, diallyl maleate, divinyl ether, diallyl monoethyleneglycol citrate, ethyleneglycol vinyl allyl citrate, allyl vinyl maleate, diallyl itaconate, ethyleneglycol diester of itaconic acid, divinyl sulfone, hexahydro 1,3,5-triacryltriazine. triallyl phosphite, diallyl ether of benzene phosphonic acid, maleic anhydride triethylene glycol polyester, polyallyl sucrose, polyallyl glucose, sucrose diacrylate, glucose dimethacrylate, pentaerythritol di-, tri- and tetra-acrylate or methacrylate, trimethylol propane di- and triacrylate or methacrylate, sorbitol dimethacrylate, 2-(1-aziridinyl)-ethyl methacrylate, tri-ethanolamine diacrylate or dimethacrylate, triethanolamine triacrylate or trimethacrylate, tartaric acid dimethacrylate, triethyleneglycol dimethacrylate, the dimethacrylate of bis-hydroxy ethylacetamide and mixtures thereof.

Other suitable polyethylenically unsaturated cross-linking monomers include ethylene glycol diacrylate, diallyl phthalate, trimethylolpropanetrimethacrylate, polyvinyl and polyallyl ethers of ethylene glycol, of glycerol, of pentaerythritol, of diethyleneglycol, of monothio- and dithio-derivatives of glycols, and of resorcinol; divinylketone, divinylsulfide, allyl acrylate, diallyl fumarate, diallyl succinate, diallyl carbonate, diallyl malonate, diallyl oxalate, diallyl adipate, diallyl sebacate, diallyl tartrate, diallyl silicate, triallyl tricarballylate, triallyl aconitrate, triallyl citrate, triallyl phosphate, divinyl naphthalene, divinylbenzene, trivinylbenzene; alkyldivinylbenzenes having from 1 to 4 alkyl groups of 1 to 2 carbon atoms substituted on the benzene nucleus; alkyltrivinylbenzenes having 1 to 3 alkyl groups of 1 to 2 carbon atoms substituted on the benzene nucleus; trivinylnaphthalenes, polyvinylanthracenes, and mixtures thereof. In addition, acryl or methracryl-encapped siloxanes and polysiloxanes, methacryloyl end-capped urethanes, urethane acrylates of polysiloxane alcohols and bisphenol A bis methacrylate and ethoxylated bisphenol A bis methacrylate also are suitable as polyunsaturated monomers.

Still another group of monomers is represented by di- or poly-vinyl ethers of ethylene, propylene, butylene, and the like, glycols, glycerin, pentaerythritol, sorbitol, di- or poly-allyl compounds such as those based on glycols, glycerin, and the like, or combinations of vinyl ally or vinyl acryloyl compounds such as vinyl methacrylate, vinyl acrylate, allyl methacrylate, allyl acrylate, methallyl methacrylate, or methallyl acrylate. In addition, aromatic, cycloaliphatic and heterocyclic compounds are suitable for this invention. These compounds include divinyl benzene, divinyl toluene, divinyl diphenyl, divinyl cyclohexane, trivinyl benzene, divinyl pyridine, and divinyl piperidine. Furthermore, divinyl ethylene or divinyl propylene urea and similar compounds may be used, e.g., as described in U.S. Pat. Nos. 3,759,880; 3,992,562; and 4,013,825. Acryloyl- or methacryloyl end-capped siloxane and polysiloxanes such as those described in U.S. Pat. No. 4,276,402 (equivalent to German Patent Publication No. 30 34 505); U.S. Pat. Nos. 4,341,889; and 4,277,595 (equivalent to French Patent 2,465,236) are suitable for this invention. Methacryloyl end-capped urethanes, such as those described in U.S. Pat. Nos. 4,224,427; 4,250,322; 4,423,099; and 4,038,257 (equivalent to German Patent Publication No. 25 42 314), German Patent Publications No. 23 65 631, Japanese Patent Publication Nos. 60-233,110; 61-009,424, and 61-030,566, and British Patent Publication No. 1,443,715, are suitable for this invention. Urethane acrylates of polysiloxane alcohols as described in U.S. Pat. Nos. 4,543,398 and 4,136,250 and bisphenol A-bis methacrylate and ethoxylated-bisphenol A-bis methacrylate are also suitable monomers for this invention. Each of the above listed patents is incorporated herein by reference.

Monoethylenically unsaturated monomers are also suitable, in an amount up to 40% by weight, and in other embodiments no more than 9% by weight, based on the total weight of monomers, for preparing polymer micro-particles include ethylene, propylene, isobutylene, disobutylene, styrene, vinyl pyridine ethylvinylbenzene, vinyltoluene, and dicyclopentadiene; esters of acrylic and methacrylic acid, including the methyl, ethyl, propyl, isopropyl, butyl, sec-butyl, tert-butyl, amyl, hexyl, octyl, ethylhexyl, decyl, dodecyl, cyclohexyl, isobornyl, phenyl, benzyl, alkylphenyl, ethoxymethyl, ethoxyethyl, ethoxyproyl, propoxymethyl, propoxyethyl, propoxypropyl, ethoxyphenyl, ethoxybenzyl, and ethoxycyclohexyl esters; vinyl esters, including vinyl acetate, vinyl propionate, vinyl butyrate and vinyl laurate, vinyl ketones, including vinyl methyl ketone, vinyl ethyl ketone, vinyl isopropyl ketone, and methyl isopropenyl ketone, vinyl ethers, including vinyl methyl ether, vinyl ethyl ether, vinyl propyl ether, and vinyl isobutyl ether; and the like.

Other monounsaturated monomer materials which may be utilized in accordance with the present invention, in an amount up to 40% by weight or less, in other embodiments no more than 25% by weight, and in other embodiments no more than 9% by weight, based on the total weight of monomers in the monomer solution, include hydroxy alkyl esters of alpha, beta-unsaturated carboxylic acids such as 2-hydroxy ethylacrylate or methacrylate, hydroxypropylacrylate or methacrylate and the like. Many derivatives of acrylic or methacrylic acid other than the esters mentioned are also suitable as starting monounsaturated monomer materials for use in forming the unsaturated polymer micro-particles of the present invention. These include, but are not limited to the following monomers: methacrylylglycolic acid, the monomethacrylates of glycol, glycerol, and of other polyhydric alcohols, the monomethacrylates of dialkylene glycols and polyalkylene glycols, and the like. The corresponding acrylates in each instance may be substituted for the methacrylates. Examples include the following: 2-hydroxyethyl acrylate or methacrylate, diethylene glycol acrylate or methacrylate, 2-hydroxypropyl hydroxypropyl acrylate or methacrylate, 3-hydroxypropyl acrylate or methacrylate, tetraethyleneglycol acrylate or methacrylate, pentaethyleneglycol acrylate or methacrylate, dipropyleneglycol acrylate or methacrylate, acrylamide, methacrylamide, diacetone acrylamide, methylolacrylamide, methylolmethacrylanide and any acrylate or methacrylate having one or more straight or branched chain alkyl groups of 1 to 30 carbon atoms, or in certain embodiments 5 to 18 carbon atoms, and the like. Other suitable examples include isobornyl methacrylate, phenoxyethyl methacrylate, isodecyl methacrylate, stearyl methacrylate, hydroxypropyl methacrylate, cyclohexyl methacrylate, dimethylaminoethyl methacrylate, t-butylaminoethyl methacrylate, 2-acrylamido propane sulfonic acid, 2-ethylexyl methacrylate, methyl methacrylate, ethyl methacrylate, butyl methacrylate, 2-hydroxyethyl methacrylate, tetrahydrofurfuryl methacrylate and methoxyethyl methacrylate.

Examples of monounsaturated monomers containing carboxylic acid groups as functional groups and suitable for use as starting materials in accordance with the invention include the following: acrylic acid, methacrylic acid, itaconic acid, aconitic acid, cinnamic acid, crotonic acid, mesaconic acid, maleic acid, fumaric acid and the like.

Partial esters of the above acids are also suitable as monounsaturated monomers for use in accordance with the invention. Examples of such esters include the following: mono-2-hydroxypropyl aconitate, mono-2-hydroxyethyl maleate, mono-2-hydroxypropyl fumarate, mono-ethyl itaconate, monomethyl cellosolve ester of itaconic acid, monomethyl cellosolve ester of maleic acid, and the like.

Examples of suitable monounsaturated monomers containing amino groups as functional groups include the following: diethylaminoethyl acrylate or methacrylate, dimethylaminoethyl acrylate or methacrylate, monoethylaminoethyl acrylate or methacrylate, tert-butylaminoethyl methacrylate, para-amino styrene, ortho-amino styrene, 2-amino-4-vinyl toluene, piperidinoethyl methacrylate, morpholinoethyl methacrylate, 2-vinyl pyridine, 3-vinyl pyridine, 4-vinyl pyridine, 2-ethyl-5-vinyl pyridine, dimethyl aminopropyl acrylate and methacrylate, dimethylaminoethyl vinyl ether, dimethylaminoethyl vinyl sulfide, diethylaminoethyl vinyl ether, aminoethyl vinyl ether, 2-pyrrolidinoethyl methacrylate, 3-dimethylamino ethyl-2-hydroxy-propylacrylate or methacrylate, 2-aminoethyl acrylate or methacrylate, isopropyl methacrylamide, N-methyl acrylamide or methacrylamide, 2-hydroxyethyl acrylamide or methacrylamide, 1-methacryloyl-2-hydroxy-3-trimethyl ammonium chloride or sulfomethylate, 2-(1-aziridinyl)-ethyl methacrylate, and the like. Polyethylenically unsaturated monomers which ordinarily act as though they have only one unsaturated group, such as isopropene, butadiene and chloroprene, should not be calculated as part of the polyunsaturated monomer content, but as part of the monoethylenically unsaturated monomer content.

In some embodiments, the porous cross-linked polymer comprises a dimethiconol cross-polymer, for example, dimethiconol/silsesquioxane copolymer, trimethylsiloxysilicate/dimethiconol cross-polymer, or a dimethiconol/acrylate copolymer.

Porous cross-linked polymers among those useful herein are disclosed in U.S. Pat. Nos. 5,955,552 and 6,387,995. Such polymers include those commercially available as: MICROSPONGE™ 5640, marketed by A.P. Pharma, Redwood City, Calif., U.S.A.; POLYTRAP™ 6603 and POLY-PORE™ 200 series, marketed by Amcol International Corp, Arlington Heights, Ill., U.S.A.; and DSPCS-12 series and SPCAT-12 series, marketed by Kobo Products, Inc., South Plainfield, N.J., U.S.A. Each of the above-listed patents is incorporated herein by reference.

Hydrophilic organic polymers useful herein include polyethylene glycols, nonionic polymers of ethylene oxide, block copolymers of ethylene oxide and propylene oxide, carboxymethylene polymers, polyvinyl pyrrolidone (PVP) and mixtures thereof. Nonaqueous hydrophilic polymers useful in the practice of the present invention in certain embodiments provide a viscosity for the composition in the amount of about 10,000 mPa-s (centipoise or cP) to 600,000 mPa-s (cP).

Hydrophilic polymers also include polymers of polyethylene glycols and ethylene oxide having the general formula: HOCH₂(CH₂OCH₂)ₙOH, wherein n represents the average number of oxyethylene groups. Polyethylene glycols available from Dow Chemical (Midland, Mich.) are designated by number such as 200, 300, 400, 600, 2000 which represents the approximate weight average molecular weight of the polymer. Polyethylene glycols 200, 300, 400, and 600 are clear viscous liquids at room temperature, and are used in certain embodiments of the present invention.

Another hydrophilic polymer useful herein is comprised of a water soluble, nonionic block copolymer of ethylene oxide and propylene oxide of the formula: HO(C₂H₄O)(C₃H₆O)_{b}(C₂H₄O)CH. The block copolymer in certain embodiments is chosen (with respect to a, b and c) such that the ethylene oxide constituent comprises about 65 to about 75% by weight, of the copolymer molecule and the copolymer has a weight average molecular weight of about 2,000 to about 15,000, with the copolymer being present in oral care composition in such concentration that the composition is liquid at room temperature (25° C.).

A block copolymer useful herein is PLURAFLO™ L1220 of BASF Corporation, which has a weight average molecular weight of about 9,800. The hydrophilic poly(ethylene oxide) block averages about 65% by weight of the polymer.

Organic polymers useful as adhesion enhancing agents include hydrophilic polymers such as carbomers such as carboxymethylene polymers such as acrylic acid polymers, and acrylic acid copolymers. Carboxypolymethylene is a slightly acidic vinyl polymer with active carboxyl groups. A carboxypolymethylene is CARBOPOL™ 974 marketed by Noveon, Inc., Cleveland, Ohio, U.S.A.

In some embodiments, a hydrophobic adhesion agent is present. Hydrophobic organic materials useful as adhesion enhancing agents in the practice of the present invention include hydrophobic materials such as waxes such as bees wax, mineral oil, mineral oil and polyethylene blends, petrolatum, white petrolatum, liquid paraffin, butane/ethylene/styrene hydrogenated copolymer) blends (VERSAGEL™ marketed by Penreco, Houston, Tex., U.S.A.), acrylate and vinyl acetate polymers and copolymers, polyethylene waxes, silicone polymers as discussed further herein and polyvinyl pyrrolidone/vinyl acetate copolymers. In some embodiments the hydrophobic adhesion agent comprises from 1% to 50% by weight of the composition, e.g., from 10-40% by weight, or from 20%-30% by weight, or about 30% by weight.

Nonionic surfactants useful in the compositions of the present invention include compounds produced by the condensation of alkylene oxides (especially ethylene oxide) with an organic hydrophobic compound, which may be aliphatic or alkylaromatic in nature. One group of surfactants is known as “ethoxamers”. These include condensation products of ethylene oxide with fatty acids, fatty alcohols, fatty amides, polyhydric alcohols, (e.g., sorbitan monostearate) and the like. “Polysorbates” is the name given to a class of nonionic surfactants prepared by ethoxylating the free hydroxyls of sorbitan-fatty acid esters. They are commercially available, for example as the TWEEN™ surfactants of ICI, US Inc. Non-limiting examples include Polysorbate 20 (polyoxyethylene 20 sorbitan monolaurate, TWEEN™ 20) and Polysorbate 80 (polyoxyethylene 20 sorbitan mono-oleate, TWEEN™ 80). In certain embodiments, polysorbates include those with about 20 to 60 moles of ethylene oxide per mole of sorbitan ester. Nonionic surfactants are optionally present in embodiments of this invention at amounts of 0.01% to 10%.

Other suitable nonionic surfactants include poly(oxyethylene)-poly(oxypropylene) block copolymers, especially triblock polymers of this type with two blocks of poly(oxyethylene) and one block of poly(oxypropylene). Such copolymers are known commercially by the non-proprietary name of poloxamers, the name being used in conjunction with a numeric suffix to designate the individual identification of each copolymer. Poloxamers may have varying contents of ethylene oxide and propylene oxide, leading to a wide range of chemical structures and molecular weights. In one embodiment, the poloxamer is poloxamer 407. It is widely available, for example under the trade name PLURONIC™ F127 of BASF Corporation.

Other non-limiting examples of suitable nonionic surfactants include products derived from the condensation of ethylene oxide with the reaction product of propylene oxide and ethylene diamine, long chain tertiary amine oxides, long chain tertiary phosphine oxides, long chain dialkyl sulfoxides and the like.

Humectants useful herein include polyhydric alcohols such as glycerin, sorbitol, xylitol or low molecular weight PEGs. In various embodiments, humectants are operable to prevent hardening of gel compositions upon exposure to air. In various embodiments humectants also function as sweeteners. One or more humectants are optionally present in a total amount of 1% to 50%, for example 2% to 25% or 5% to 15%.

The disclosure further provides in one embodiment an oral care system (System 1) comprising
a gel, e.g., a tooth whitening gel according to any of the preceding embodiments, e.g., Gel 1, et seq.,
contained in a dispenser, wherein
the dispenser (Dispenser 1) comprises: a housing having a longitudinal axis and an internal reservoir containing the gel; a dispensing orifice in the housing for dispensing the gel from the reservoir, a removable or displaceable cap which can cover the dispensing orifice when the dispenser is not in use; and means for dispensing the gel from the dispensing orifice;
for example the oral care system of System 1 comprising
1.1. Dispenser 1 wherein the means for dispensing the gel is a surface at the end of the internal reservoir which is distal to the dispensing orifice and axially movable towards the orifice, such that when the surface is moved towards the dispensing orifice, the gel is dispensed, for example wherein the surface is moved by means of external pressure or by means of a drive screw which exerts force to move the surface when the drive screw is turned;
1.2. A dispenser, e.g. according to 1 or 1.1 comprising a longitudinally elongated housing having a distal end with an applicator therein and an opposite proximal end; a reservoir disposed in the housing for holding a plaque indicator gel as hereinbefore described, the reservoir in fluid communication with the applicator;
1.3. Dispenser 1, 1.1 or 1.2 comprising a collar within the housing, the collar comprising an axial passageway and a cam surface, the collar being non-rotatable with respect to the housing; a reciprocator comprising an actuator, a drive screw extending through the axial passageway of the collar, and a cam surface, the reciprocator being rotatable with respect to the housing; a resilient member that axially biases the cam surface of the reciprocator and the cam surface of the collar into mating contact; an elevator forming an end wall of the reservoir, the elevator being non-rotatable with respect to the housing and threadily coupled to the drive screw; and wherein rotation of the actuator causes the elevator to (1) axially advance along the drive screw in a first axial direction due to relative rotation between the drive screw and the elevator, and (2) axially reciprocate due to relative rotation between the cam surface of the collar and the cam surface of the reciprocator;
1.4. Any of the foregoing dispensers wherein the dispenser forms all or part of the handle of a toothbrush, for example wherein the head portion of the toothbrush forms the cap of the dispenser, or wherein the head portion of the toothbrush can be rotated to turn a drive screw which dispenses the gel from the opposite end;
1.5. Any of the foregoing dispensers wherein the dispensing orifice comprises a brush suitable for controlled application of the gel to the teeth;
1.6. Any of the foregoing dispensers wherein the dispensing orifice is in the form of a doe foot suitable for controlled application of the gel to the teeth;
1.7. Any of the foregoing dispensers wherein the exterior surface of the dispensing orifice comprises an elastomeric material;
1.8. Any of the foregoing dispensers wherein the exterior surface of the dispensing orifice has nubbins;
1.9. Any of the foregoing dispensers wherein the gel is dispensed as a shear rate of 50-200/s, e.g., 75-125/s, e.g., about 100/s;
1.10. Any of the foregoing dispensers comprising a gel, e.g., a whitening gel according to Gel 1, et seq.

Examples of dispensers suitable for use in oral care systems according to the present invention include those more fully described, for example, in WO 2011/079028, WO/2011/078864, and WO/2011/078863, the entire contents of which are incorporated herein by reference. Particular embodiments include oral care systems utilizing dispensers having a reservoir and a dispensing orifice in the form of a brush or a doe foot, wherein the dispenser forms the handle of a toothbrush, for example where the head of the toothbrush is removed when the gel is dispensed or the head of the toothbrush is turned to dispense the gel from the opposite end.

In a further embodiment, the disclosure provides a method of whitening the teeth comprising applying a tooth whitening gel according to any of the preceding embodiments, e.g., Gel 1, et seq., in an oral care system comprising the gel in a dispenser, e.g. a dispenser according to Dispenser 1, et seq., to the teeth of a subject in need thereof, and permitting the gel to remain on the teeth for a sufficient amount of time to achieve a whitening effect, e.g., for 5 to 30 minutes, or, e.g., from 10 to 20 minutes. Also provided is the use of a tooth whitening gel according to any of the preceding embodiments, e.g., Gel 1, et seq., in such a method or in the manufacture of an oral care system for use in such a method.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

### Example 1—Gel Formulation Optimization

Different gel formulations are prepared and tested to determine suitability for administration with a pen-type dispenser. Many of the formulae tested are too runny or too thick for use with the pen dispenser. Three formulae identified as having potentially acceptable rheological properties based on the initial visual evaluation and testing with pen dispenser are selected for more detailed evaluation. Formulae A, B, and C are prepared in accordance with the following table (ingredients by weight % of total formula).

| Ingredient | A | B | C |
|---|---|---|---|
| Demineralized Water | 56.24 | 54.94 | 52.14 |
| Sodium Saccharin | 0.50 | 0.50 | 0.50 |
| Sodium Fluoride | 0.11 | 0.11 | 0.11 |
| Glycerin | 20.00 | 20.00 | 20.00 |
| Xanthan Gum | 0.20 | 0.50 | 1.50 |
| Sodium CMC-Type 7 | 0.20 | 1.20 | 3.00 |
| Sorbitol-70% solution | 20.00 | 20.00 | 20.00 |
| Sodium Lauryl Sulfate Powder | 1.50 | 1.50 | 1.50 |
| Flavoring | 1.20 | 1.20 | 1.20 |
| Coloring | 0.05 | 0.05 | 0.05 |

The formulae are compared for suitability in the intended use, testing the formulae in two different pen dispenser types, one with a doe foot tip and the other with a brush tip. The results are summarized in the following table:

| | A | B | C |
|---|---|---|---|
| Dispensing | Poor-runs off | Acceptable | Product splits brush, |
| | applicator | | unacceptable for doe |
| | | | foot-product keeps |
| | | | dispensing after |
| | | | turning applicator |
| Stand Up | Unacceptable-runs | Acceptable- | Acceptable |
| | off applicator and | stays on | |
| | brush | applicator | |
| Application | Unacceptable, runs | Spreads evenly | Unacceptable- |
| | upon application | with all | Spreading is difficult |
| | | applicators | with doe foot and brush |

The composition of Formula Bis seen to be the most suitable for this application. The critical differences between the three formulae relate to their rheological properties, as seen in the following summary table:

| Condition | Relevant Rheological Property |
|---|---|
| Dispensing | Viscosity profile G′/G″ |
| Stand Up | Critical stress |
| Application | Viscosity at shear rate of ~1000 s⁻¹ Critical stress |

The selected gels are non-Newtonian, exhibiting non-linear shear-thinning properties at different levels of force. The specific rheological properties of the formulations are measured using an AR1000 rheometer from TA Instruments with the 4 cm 2 degree cone geometry. Viscoelastic properties, such as the elastic modulus (G′) and the loss modulus (G″), are obtained from strain sweep experiments. For the strain sweep measurements, the angular frequency is held at 1 Hz while the strain is varied from 0.1 to 500%. Viscosity measurements are obtained from steady state flow experiments, which are conducted varying the shear rate from 1000 to 0.1 s⁻¹. The data is plotted into the Herschel-Bulkley (HB) Model (shear stress=yield stress+viscosity*(shear rate)^{rate index}):

| | | | Critical | | HB fit: | HB fit: | |
|---|---|---|---|---|---|---|---|
| | G′ | G″ | Stress | | yield stress | viscosity | HB fit: |
| Formula | (dyne/cm²) | (dyne/cm²) | (dyne/cm²) | G′/G″ | (dyne/cm²) | (poise) | rate index |
| A | 22.66 | 17.61 | 2.151 | 1.286 | 8.628 | 2.429 | 0.6639 |
| B | 330 | 151.3 | 5.06 | 2.181 | 37.67 | 39.32 | 0.5342 |
| C | 2170 | 909.7 | 19.39 | 2.385 | 285.3 | 631.1 | 0.3754 |

Based on the suitability and rheological data, gels for this application should have (i) HB yield stress greater than Formula A and less than Formula C, e.g., about that of Formula B, (ii) HB viscosity greater than Formula A and less than Formula C, e.g., about that of Formula B, and (iii) HB rate index less than Formula A and greater than Formula C, e.g., about that of Formula B.

### Example 2—Bleaching Time

Samples of a commercial dental whitening gel containing PVP-hydrogen peroxide as the bleaching agent, polydimethylsiloxane, and a porous cross-linked polymer, are prepared that each additionally contain 2% sodium tripolyphosphate and either 0%, 0.05%, 0.15%, 0.50%, 1.0% or 5.0% of Polysorbate-20. 0.5 g of each formulation is spread onto a glass slide and smoothed. Eight drops of 1.7 mM Lissamine Green dye solution are added to the slide and the time taken to completely bleach 75% of the drops is recorded. The results are summarized in the Table below.

| Polysorbate-20 | Bleaching |
|---|---|
| Concentration | Time |
| (% by Wt.) | (minutes) |
| 0% | 18 |
| 0.05% | 13 |
| 0.15% | 9 |
| 0.50% | 7 |
| 1.0% | 6 |
| 5.0% | 6 |

The results show that addition of from 0.05 to 5.0% by weight of Polysorbate-20 results in a significant reduction in bleaching time. In addition, it is observed that as the concentration of Polysorbate-20 is increased, the wetting angle of the drops increases. This suggests that the Polysorbate-20 increases the hydrophilicity of the composition, and without being bound by theory, it is believed that the increased hydrophilicity of the composition results in enhanced release of hydrogen peroxide.

According to one embodiment of the present invention, the following exemplary composition is provided. All amounts are in % by weight of the composition:

| Material | Range | Example |
|---|---|---|
| Polydimethylsiloxane | 20-40% | 31% |
| Mineral oil | 20-40% | 27% |
| Polyvinylpyrrolidone* | 10-30% | 20% |
| Hydrogen Peroxide* | 2-6% | 4.5% |
| Trimethylsiloxysilicate/ | 5-20% | 11% |
| dimethiconol cross-polymer | | |
| Polyethylene | 0-5% | 1.5% |
| Flavoring | 0-3% | 0.6% |
| Sweetener | 0-3% | 0.3% |
| Sodium | 0-5% | 2% |
| tripolyphosphate | | |
| Polysorbate-20 | 0.1-5% | 1% |

*Provided as PVP-hydrogen peroxide complex

What is claimed is:

1. An orally acceptable tooth whitening gel comprising:
(i) a hydrogen peroxide source,
(ii) a silicone compound,
(iii) a porous cross-linked polymer, and
(iv) a nonionic surfactant,
the gel having a Herschel-Bulkley yield stress of 10 to 230 dynes/cm², e.g., 30 to 45 dynes/cm², a Herschel-Bulkley viscosity of 3 to 500 poise, e.g., 30 to 45 poise, and a Herschel-Bulkley rate index of 0.4 to 0.6, e.g. 0.5 to 0.6.

2. The gel of claim 1, wherein the hydrogen peroxide source is a hydrogen peroxide-polymer complex.

3. The gel of claim 1, wherein the hydrogen peroxide source is a polyvinylpyrrolidone-hydrogen peroxide complex

4. The gel of claim 1, wherein the gel contains 0.1-10% by weight of hydrogen peroxide, e.g., 0.5-10% by weight, or 1-5% by weight, or 2-5% by weight, or 3-5% by weight, or 4-5% by weight, or about 4.5% by weight of hydrogen peroxide.

5. The gel of claim 1, wherein the silicone compound comprises a silicone polymer, silicone adhesive, silicone gum, silicone wax, silicone elastomer, silicone fluid, silicone resin, silicone powder, or mixture thereof.

6. The gel of claim 1, wherein the silicone compound comprises a polydiorganosiloxane.

7. The gel of claim 1, wherein the silicone compound is a polydimethylsiloxane.

8. The gel of claim 1, wherein the silicone compound is present in the composition in an amount of 10 to 50% by weight, e.g., 20-40%, or about 30%.

9. The gel of claim 1, further comprising a hydrophilic organic polymer.

10. The gel of claim 9, wherein the hydrophilic organic polymer is selected from a polyethylene glycol, a nonionic polymer of ethylene oxide, a block copolymer of ethylene oxide and propylene oxide, a carboxymethylene polymer, polyvinylpyrrolidone, and mixtures thereof.

11. The gel of claim 10, wherein the hydrophilic organic polymer is polyvinylpyrrolidone.

12. The gel of claim 1, wherein the nonionic surfactant is selected from a polyoxyethylene sorbitan monolaurate (polysorbate) or a poly(oxyethylene)-poly(oxypropylene) block copolymers (poloxamers).

13. The gel of claim 1, wherein the nonionic surfactant is a Polysorbate 20 or Polysorbate 80.

14. The gel of claim 1, wherein the nonionic surfactant is present at 0.01 to 10% by weight of the composition, e.g., 0.05 to 5%, or 0.15 to 1%, or 0.5 to 1%, or about 1% by weight of the composition.

15. The gel of claim 1, wherein the porous cross-linked polymer comprises at least one polymerized polyunsaturated monomer chosen from acrylate and methacrylate or where the porous cross-linked polymer comprises a polyitaconate or a dimethiconol cross-polymer.

16. The gel of claim 1, wherein the porous cross-linked polymer comprises a dimethiconol cross-polymer, for example, dimethiconol/silsesquioxane copolymer, trimethylsiloxysilicate/dimethiconol cross-polymer, or dimethiconol/acrylate copolymer.

17. The gel of claim 1, wherein the porous cross-linked polymer has a BET pore volume of 0.1 to 0.3 cc/g.

18. A method of whitening the teeth comprising applying a tooth whitening gel according to claim 1 to the teeth of a subject in need thereof, and permitting the gel to remain on the teeth for a sufficient amount of time to achieve a whitening effect, e.g., for 5 to 30 minutes, or, e.g., from 10 to 20 minutes.
